(19) United States
(12) Patent Application Publication
(10) Pub. No.: US 2013/0046205 A1
(43) Pub. Date: Feb. 21, 2013

(54) DEVICE AND METHOD FOR DETECTING AND MEASURING PAIN

(71) Applicants: Nils Schaffner, Luzern (CH); Gerd Folkers, Zurich (CH); Bernard Brinkhaus, Oetwil a.d. Limmat (CH); Marco Schuurmans Stekhoven, Zurich (CH)

(72) Inventors: Nils Schaffner, Luzern (CH); Gerd Folkers, Zurich (CH); Bernard Brinkhaus, Oetwil a.d. Limmat (CH); Marco Schuurmans Stekhoven, Zurich (CH)

(73) Assignee: MSYS AG, Zurich (CH)

(21) Appl. No.: 13/519,866

(22) PCT Filed: Dec. 24, 2010
(86) PCT No.: PCT/EP2010/070736

§ 371 Date: Nov. 7, 2012

(30) Foreign Application Priority Data
Dec. 28, 2009 (EP) PCT/EP2009/067955
Feb. 7, 2010 (EP) PCT/EP2010/051455

(51) Int. Cl. A61B 5/103

(52) U.S. Cl. 600/587

(57) ABSTRACT

The invention relates to a device (1) for detecting and measuring pain felt by a person, said device comprising a pressure or force sensor (2), a hollow body (3) having an outer sleeve (3a) and an inner space (3b), and an electronic unit (5) for detecting the signal of the pressure or force sensor (2). The outer sleeve (3a) of the hollow body (3) is embodied in such a way that it can be at least partially surrounded by a hand, the inner space (3b) of the hollow body (3) is filled with a non-gaseous elastic material (4a) or a non-gaseous fluid (4b), and the pressure or force sensor (2) is arranged in such a way that the pressure of the elastic material (4a) or the fluid (4b) can be measured.

The invention relates to an apparatus for detecting and measuring pain in accordance with the preamble of claim 1. The invention further relates to an apparatus for detecting and measuring pain in accordance with the preamble of claim 20.

### PRIOR ART

Pain is one of the most frequent reasons for consulting a physician. The patient often cannot give a clearly understandable answer to the physician's question of how severe the pain is. Either the patient cannot find the right words or the physician does not understand the words used. This is inter alia due to the fact that pain is a subjective sense impression which includes sensory, cognitive and emotional aspects. An accurate measurement of pain is, however, very important, e.g. for diagnosis, when adjusting the medicinal pain therapy or in pain medication research. A plurality of methods of detecting pain have been developed to date. Most of them can be divided into two categories: One-dimensional pain scales and multidimensional pain questionnaires. The most important one-dimensional scale is the “Visual Analog Scale” (VAS). It consists of a 10 cm long line whose left end is marked by “No pain” and whose right end is marked by “”Very severe pain“. The patient marks the point on the line corresponding to his pain. The NRS (Numerical Rating Scale), where the selection of a number from 0-10 is communicated verbally to represent the pain, is closely related to the VAS. The VAS is used the most since it is not at all complicated and does not require any great explanations. The problem with such scales is, however, that as a person completing the scale one generally tends toward the center and is averse to marking the extremes. On a multiple use of the VAS, users also tend toward an evaluation which becomes closer and closer (convergence) with different pain intensity. This has the consequence that the physician acquires the impression with the detection method that only small changes are present. The most important pain questionnaire is the McGill Pain Questionnaire (MPQ). The patient is given a large choice of adjectives of which he checks the ones which correspond to his pain. The adjectives are divided into three classes: sensory, affective and evaluative. A plurality of aspects of the pain are thus admittedly detected, but filling out takes a long time and the patient also has to understand and/or know all the adjectives to be able to complete the MPQ correctly.

There are various technical devices to detect pain, for example the apparatus disclosed in the document EP 0 874 587 B1. They are, however, mostly computerized variants of the known scales VAS or questionnaires MPQ, including their disadvantages. There are furthermore devices for applying pain stimuli for the measurement of pain thresholds and pain tolerance, e.g. the apparatus disclosed in the document WO 2004/103230.

Document WO 2009/052100 discloses a further apparatus for measuring pain. This apparatus has the disadvantage that the pain can only be measured with extreme imprecision and that the apparatus is only suitable to measure intestinal pain.

### REPRESENTATION OF THE INVENTION

It is the object of the present invention to form a more advantageous apparatus for detecting pain.

This object is satisfied by an apparatus comprising the features of claim 1. Dependent claims 2 to 18 relate to further advantageous embodiments. This object is further satisfied by a method comprising the features of claim 20. Dependent claims 21 to 31 relate to further advantageous method steps.

The object is in particular satisfied by an apparatus for detecting and measuring pain of a person comprising a pressure sensor or force sensor, comprising a hollow body having an outer sleeve, in particular an elastic outer sleeve and an inner space, wherein the outer sleeve at least partly bounds the inner space, and also comprising an electronic unit for detecting the signal of the pressure sensor or force sensor, wherein the outer sleeve of the hollow body is designed such that it can be at least partly surrounded by a hand, and wherein the inner space of the hollow body is filled with an elastic material, which is in particular not gaseous, or with a fluid, which is in particular not gaseous, and wherein the pressure sensor or force sensor is arranged such that the pressure of the elastic material or of the fluid can be measured.

The object is further in particular satisfied by a method of detecting and measuring pain of a person in that a pressure is exerted onto an outer sleeve, in particular an elastic outer sleeve, of a hollow body by a hand, in particular by pressing the hand closed, and in that a pressure or a force in the hollow body is measured, wherein the measured pressure or the measured force is used as a measure for the pain perceived by the person.

The apparatus in accordance with the invention relates to the field of pain measurement and its detection systems. The results of such pain measurements serve for diagnosis, research and finding relief possibilities.

The apparatus in accordance with the invention makes it possible to detect pain and preferably also to associate the pain with specific pain levels. In an advantageous embodiment, the apparatus in accordance with the invention also makes it possible to generate pain. A substantial advantage of the apparatus in accordance with the invention can be seen in the fact that it makes it possible to measure pain objectively and preferably to order its amount in specific levels. A person has the nature that he pulls back the hand by way of a reflex on pain, in particular on severe pain, with the palm of the hand being able to be contracted until a first is formed. To make use of this reflexive behavior of people, the apparatus in accordance with the invention is designed such that it has a hollow body which can be surrounded at least partly by a hand. The hollow body is designed in tubular form, in particular as a tube extending in a straight line, in a particularly advantageous embodiment. A hollow body of such a design sits easily in the hand and utilizes the natural, reflexive movement of the person to measure pain in that the hand holds the hollow body more strongly and more powerfully in a natural manner as the pain increases. The surface of the hollow body which is designed to contact the hand should preferably designed to be shape-stable, or substantially shape-stable, which produces the advantage that its shape does not change, or only changes slightly, independently of the engaging force of the hand. This allows the force effected by the hand to be measured in a reproducible and accurate manner. In other words, if the shape of the hollow body were to change due to the force effected on the hollow body by the hand, which would be the case, for example, with a rubber bellows filled with air, the perceived pain would no longer be measurable via the force effected by the hand since the rubber bellows yields as the force increases and changes its shape so that it is difficult or is no longer possible to express the perceived pain in a preferably linear manner via the force, in particular when the hand is already clenched to a fist. It can prove to be advantageous to design the surface of the hollow body such that no pronounced, and possibly even painful, pressure spots are formed at the hand surface which contacts the apparatus in accordance with the invention. Such pressure points are unpleasant and could even falsify the measurement. In an advantageous embodiment, the surface of the hollow body which is designed for contact with the hand has a specific elasticity to avoid such pressure points or pain points at the contacting hand. The apparatus in accordance with the invention makes it possible to measure the pressure or pressing force effected by the hand particularly accurately. The inner space of the hollow body is in this respect preferably filled with an elastic, non-gaseous material or with a non-gaseous fluid, with a pressure sensor or force sensor measuring the pressure of the elastic material or of the fluid which was generated by the hand engaging at the apparatus. The apparatus in accordance with the invention allows the severity of the perceived pain to be measured reliably and also reproducibly on an individual person.

In particular a material such as silicone, rubber, vulcanized rubber or a gel is suitable as an elastic material. Such materials are usually not flowable.

The inner space can, however, also be filled with a non-gaseous fluid such as a liquid or a gel to transmit the force engaging at the outer sleeve of the hollow body reliably to the pressure sensor or force sensor. The apparatus in accordance with the invention whose inner space is filled with such a material has the advantage that the engaging force can be transmitted free of hysteresis or with very small hysteresis to the pressure sensor or force sensor, which has the consequence that the engaging force and also small changes in the force can be measured very accurately so that, for example, also small changes in the force and in particular also a falling force, in particular a slightly falling force, can be measured very accurately. The apparatus in accordance with the invention thus makes it possible to measure accurately, reproducibly and in particular with small hysteresis or even without hysteresis both preferably the absolute force and force changes which are effected by the hand.

The apparatus in accordance with the invention in particular has the advantage that the pain, and preferably also its states, can be detected exactly and that the measurements of the pain are objectively reproducible.

The invention will be described in the following with reference to embodiments.

### BRIEF DESCRIPTION OF THE DRAWINGS

The drawings used to illustrate the embodiments show:

FIG. 1 schematically, a longitudinal section through an embodiment of an apparatus for detecting pain;

FIG. 2 schematically, a side view of a further embodiment of an apparatus for detecting pain;

FIG. 3 schematically, a side view of a further embodiment of an apparatus for detecting pain;

FIG. 4 schematically, a section through a housing with an integrated pressure sensor or force sensor:

FIG. 5 schematically, a side view of a further embodiment of an apparatus for detecting pain;

FIG. 6 the results of a known apparatus for measuring pain;

FIG. 7 the results of an apparatus in accordance with the invention for measuring pain;

FIG. 8 an example of a calibration of the apparatus in accordance with the invention;

FIG. 9 schematically, a further embodiment of an apparatus for detecting pain;

FIG. 10 schematically, a further embodiment of an apparatus for detecting pain;

FIG. 11 a longitudinal section through a further embodiment of an apparatus for detecting pain along the line B-B;

FIG. 12 a cross-section along the line A-A through the apparatus shown in FIG. 11;

FIG. 13 a longitudinal section through a further embodiment of an apparatus for detecting pain along the line D-D; and

FIG. 14 a cross-section along the line C-C through the apparatus shown in FIG. 13.

Generally, the same parts are provided with the same reference numerals in the drawings.

### WAYS OF CARRYING OUT THE INVENTION

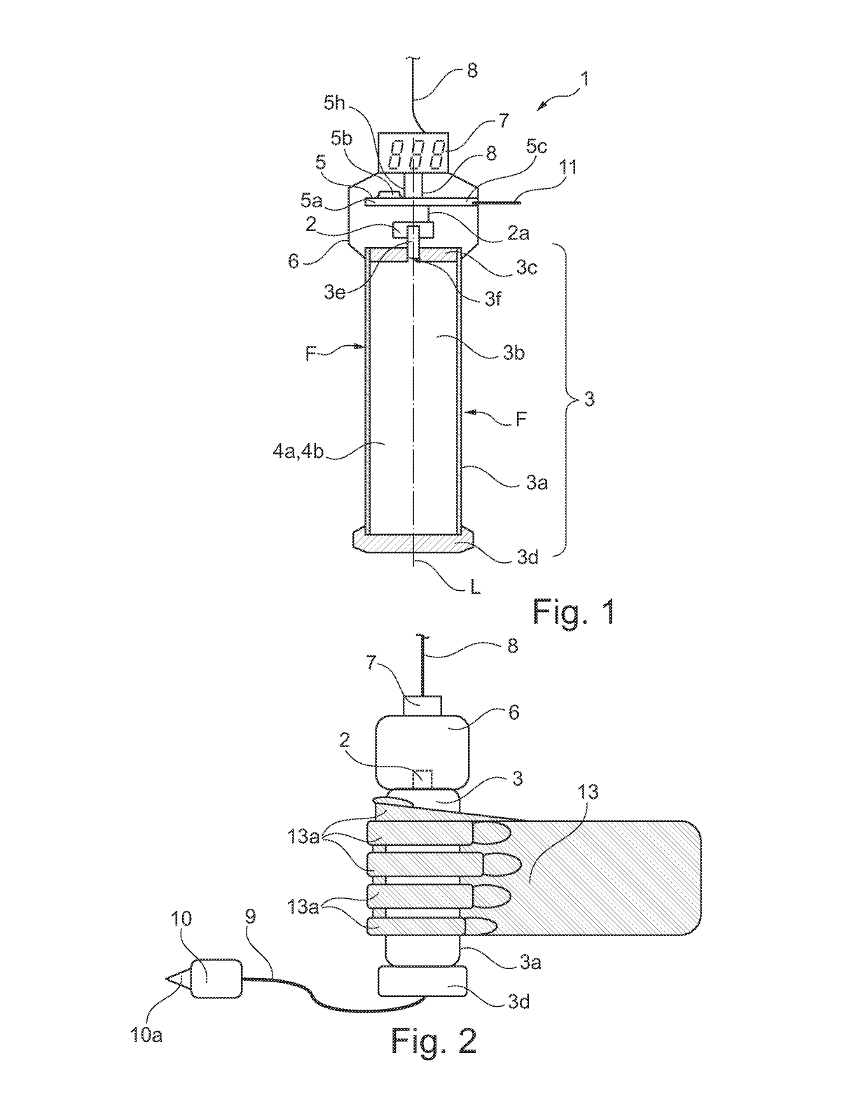

FIG. 1 shows an apparatus 1 for detecting pain in a schematic longitudinal section. The apparatus 1 for detecting pain comprises a hollow body 3 having an elastic outer sleeve 3a which bounds an inner space 3b as shown at least partly. The elastic outer sleeve 3a could be made of silicone, vulcanized rubber or rubber, for example. In the embodiment shown, the outer sleeve 3a is connected to an upper end part 3c as well as to a lower end part 3d, with the upper end part 3c, the lower end part 3d and the outer sleeve 3a bounding the inner space 3b so that the inner space 3b is preferably fluid-tight. The upper and lower end parts 3c, 3d could also be designed as elastic. In a preferred embodiment, the upper and lower end parts 3c, 3d are, however, designed as rigid and are made, for example, from a plastic or from metal. In the embodiment shown, the outer sleeve 3a is designed in the shape of a hollow cylinder, with the upper and lower end parts 3c, 3d being designed in the shape of a circular disk, so that the hollow body 3 has a longitudinal axis L which moreover forms an axis of symmetry with respect to the hollow body 3 in an advantageous embodiment. The upper end part 3c has a circular opening 3f at the center, starting from which a tubular extension 3e extends in the direction of extent of the longitudinal axis L. A pressure sensor or force sensor 2 is arranged at the end of the tubular extension. The inner space 3b of the hollow body 3 as well as the tubular extension 3e are filled completely with an elastic material 4a, not shown as visible, or with a fluid 4b. The inner space 3b is particularly advantageously filled with an elastic, non-gaseous material 4a or with a non-gaseous fluid 4b. This has the consequence that, for example, a compression force F exerted onto the outer sleeve 3a can be detected by the pressure sensor or force sensor 2 since the pressure increase effected due to the compression force F in the inner space 3b is transmitted from the elastic material 4a or from the fluid 4b onto the pressure sensor or force sensor 2. A non-gaseous fluid such as a liquid or a gel is suitable as a fluid. In a preferred embodiment, the elastic material 4a has a lower elasticity than the outer sleeve 3a so that the outer sleeve 3a feels pleasingly elastic and the force F can thus be introduced particularly easily into the elastic material 4a. As shown in FIG. 1, the elastic outer sleeve 3a forms the division between the inner space 3b and the outer space along the longitudinal direction L, starting at the upper end part 3c and ending at the lower end part 3d. The diameter of the hollow body 3 is selected such that the elastic outer sleeve 3a can be at least partly gripped around by a hand 13 so that the hand 13 can be placed pleasingly onto the elastic outer sleeve 3a and the hand 13 partly or completely surrounds the elastic outer sleeve 3a in the peripheral direction. When the hand 13 is pressed together, a force F is effected onto the elastic outer sleeve 3a, which has the consequence that the pressure of the elastic material 4a or of the fluid 4b located in the inner space increases. The pressure sensor or force sensor 2 can thus measure the pressure in the inner space in mbar or Newton, for example. In a particularly advantageous embodiment, at least that part of the surface of the apparatus 1 in accordance with the invention which is held by the hand during the measurement is designed as an elastic outer sleeve 3a, with the elastic outer sleeve 3a, as shown in FIG. 1, directly bounding the inner space 3b from the outer space. If the hand only contacts the elastic outer sleeve 3a during the compression or during the measurement of the pain, this produces the advantage that the hand does not contain any pressure points, which could falsify the pain measurement. If the hand were, for example, partly to contact the housing 6 and/or the upper end part 3c and/or the lower end part 3d during the compression, the patient would reduce the compression force reflexively on the compression to avoid any pain and/or unpleasant feelings occurring at the hand. The elastic outer sleeve 3a designed and arranged in this manner, in combination with the elastic material 4a or with the fluid 4b located in the inner space 3b and in combination with the pressure sensor or force sensor 2 which detects the pressure in the inner space 3b, makes it possible to measure the pressing force of the hand reproducibly and exactly so that the subjectively perceived pain can be measured which is expressed by a patient via the pressing force of the hand. Investigations with patients have surprisingly shown that such an apparatus is suitable to measure subjectively perceived pain reproducibly via the pressing force of the hand.

The apparatus 1 shown in FIG. 1 moreover includes a housing 6 which is fastened to the upper end part 3c, on the one hand, and which is fastened to a display apparatus 7, on the other hand. An electronic unit 5 is arranged within the housing 6 and in the embodiment shown includes a circuit board 5a, electronic modules 5b as well as a read/write apparatus 5c for a memory card 11. The memory card 11 is preferably, as shown in FIG. 1, insertable into the electronic unit 5 from the outside and is preferably also replaceable. The electronic unit 5 is connected to the display apparatus 7 via signal lines. In addition, the electronic unit 5 is connected to the pressure sensor or force sensor 2 via a signal line 2b. In addition, the electronic unit 5 is connected via a cable 8 to an apparatus arranged outside the apparatus 1. The cable 8 can serve, for example, for the energy supply and/or for transmitting signals or data. The electronic modules 5b could moreover include an evaluation apparatus 5f, not shown, and/or an acoustic output apparatus 5g.

In a further possible embodiment, the upper and lower end parts 3c, 3d could also be mutually directly connected to one another, for example by a connection bar which extends in the direction of the longitudinal axis L in the inner space 3b and which mutually fixedly connects the two end parts 3c, 3d.

FIG. 2 shows a further embodiment of an apparatus 1 for detecting and measuring pain in a side view. The apparatus 1 comprises a hollow body 3 having an elastic outer sleeve 3a, with the hollow body 3, in the same way as shown in FIG. 1, being bounded at the top and bottom by an upper end part 3c and by a lower end part 3d. The upper part 3c and the pressure sensor or force sensor 2 shown by dashed lines are arranged within the housing 6. A display 7 is moreover arranged at the housing 6. In addition, a connection cable 8 as described in FIG. 1 is provided. FIG. 2 moreover schematically shows a hand 13 with fingers 13a, with the fingers 13a surrounding the elastic outer sleeve 3a. The force effected on the elastic outer sleeve 3a by the hand 13 can be detected by the pressure sensor or force sensor 2.

The apparatus 1 in accordance with the invention can moreover include a plan actuator 10 which is connected to the apparatus 1 via an electric line 9, for example. The pain actuator 10 includes an apparatus 10a to generate controlled pain, for example a needle or a warmable or heatable contact surface such as a hot tip.

The apparatus in accordance with the invention 1 can moreover include at least one additional sensor 14 for detecting physiological values, in particular a sensor for detecting the skin conductivity, or a sensor for detecting the pulse or a chemical sensor for detecting biological values. The additional sensor 14 can, as shown in FIG. 2, be arranged, for example, at the surface of the elastic outer sleeve 3a or within the elastic outer sleeve 3a, preferably also such that a direct contact is possible between the hand 13 and/or fingers 13a and the sensor 14. The sensor 14 could, however, also be arranged at another point of the apparatus 1, such as in the housing 6 or at the surface of the housing 6. The measurement of physiological parameters such as the skin conductivity, blood pressure, heart rate, etc. does not per se produce any clear statement on the severity of the pain since these parameters are greatly influenced by emotional states such as nervousness and fear. These physiological parameters can, however, also serve to interpret the statements or the personal assessment of the patient in order thereby to improve the quality of the pain measurement.

The apparatus 1 shown in FIGS. 1 and 2 has the advantage that the force measured by the pressure sensor or force sensor 2 extends substantially perpendicular to the introduced force F. This configuration makes it possible to measure the force F particularly accurately and in particularly largely free of interference.

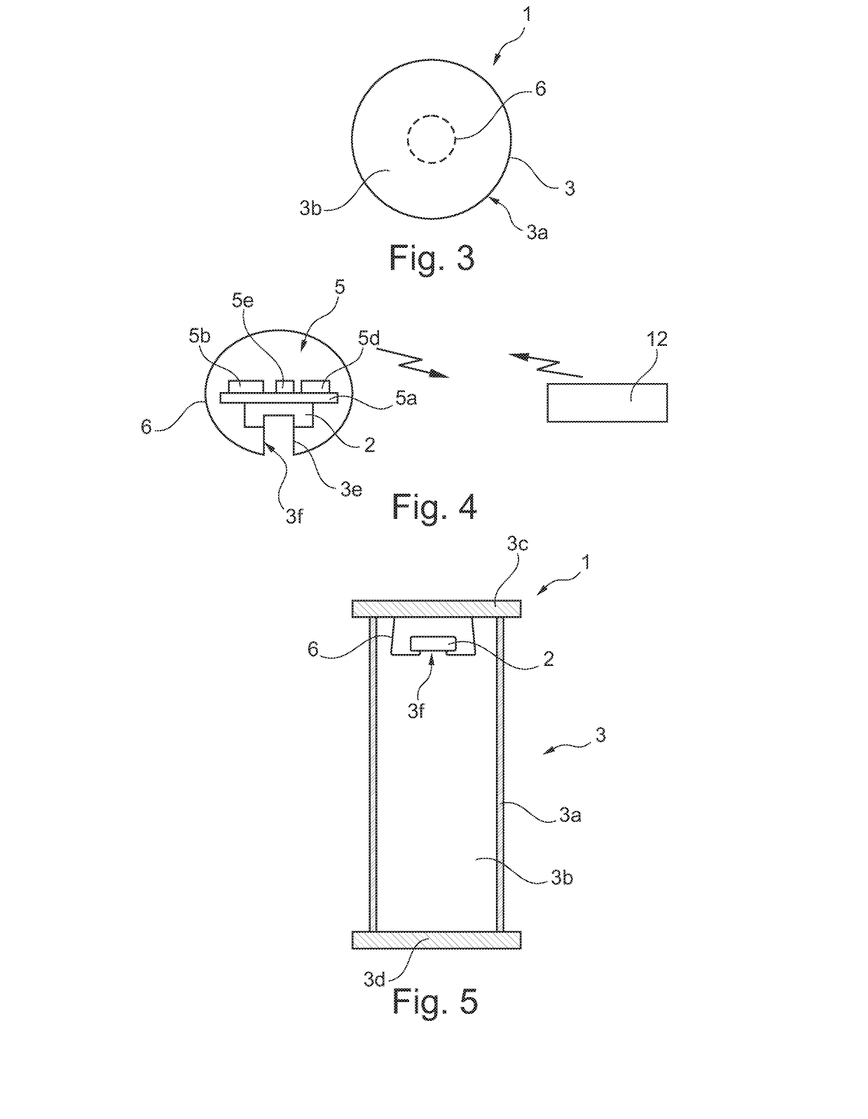

FIG. 3 schematically shows a further embodiment of an apparatus 1 for detecting and measuring pain. This apparatus 1 is designed as spherical in the embodiment shown and includes a hollow body 3 having an elastic outer sleeve 3a and an inner space 3b, with a housing 6 which is explained in detail in FIG. 4 being arranged in the inner space 3b. In addition, the inner space 3b is filled with an elastic material 4a or with a fluid 4b. The apparatus 1 or the hollow body 3 could also have a different outer shape and be designed in cube shape, for example. The housing 6 can also be designed in a plurality of possible shapes and can, for example, have a spherical or cube-shaped outer shape.

FIG. 4 schematically shows a section through the housing 6 shown in FIG. 3. The spherical housing 6 has at the outer shell a circular or cylindrical opening 3f from which a tubular extension 3e extends into the interior up to the pressure sensor or force sensor 2. The tubular extension 3e is connected to the pressure sensor or force sensor 2 in a fluid-tight manner so that no fluid can penetrate into the inner space of the housing 6. In addition, an electronic unit 5 is arranged in the inner space of the housing 6 and comprises a circuit board 5a on which electronic modules 5b, a battery 5d and a data transmission apparatus 5e are arranged. The data transmission apparatus 5e is preferably designed so that a wireless data transmission to an external transmission and reception apparatus 12 is possible. The data could, for example, be transmitted with the aid of light or of electromagnetic waves. In an advantageous embodiment, the electronic unit 5 moreover includes an acoustic output apparatus 5g, for example a small loudspeaker or a vibrator, and/or an evaluation apparatus 5f. The evaluation apparatus 5f could also be arranged in the transmission and reception apparatus 12.

FIG. 5 schematically shows a further embodiment of an apparatus 1 for detecting and measuring pain in a longitudinal section. The apparatus 1 in turn includes, in a similar manner as shown in FIG. 1, a hollow body 3 having a hollow-cylindrical, elastic outer sleeve 3a which is connected in a fluid-tight manner at the top and at the bottom to an upper and lower end part 3c, 3d so that a fluid-tight inner space 3b is formed. Unlike the embodiment shown in FIG. 1, the housing 6 and the pressure sensor or force sensor 2 are arranged completely in the interior of the inner space 3b in the embodiment shown in FIG. 5, with the housing 6 having an opening 3f at which the pressure sensor or force sensor 2 is arranged. The inner space of the housing 6 could also be designed in a similar manner as shown in FIG. 4 and could in particular also include a battery 5d and a data transmission apparatus 5e.

The apparatus 1 in accordance with the invention and in particular the extent of the outer sleeve 3a can be designed in a plurality of possibilities and shapes. The in particular elastic outer sleeve 3a can in particular also be designed in accordance with the anatomy of a hand so that the elastic outer sleeve 3a sits particularly easily in the hand 13.

A measurement of the pain using the apparatus 1 in accordance with the invention takes place such that a patient is asked to place his hand onto the hollow body 3 and then to express the felt pain intuitively by pressing the hand closed. The apparatus 1, preferably designed in tubular form, sits easily in the hand 13 in this respect. The apparatus in accordance with the invention can, as shown in FIGS. 1 and 2, be connected via a thin cable 8 to a subordinate apparatus such as a laptop computer or a palm computer. In an advantageous embodiment, the apparatus 1 in accordance with the invention includes a mass storage device such as an installed logger, and preferably a memory card such as an SD card, to store data over a longer time period so that the patient can also take measurements independently over a longer time and the data are recorded invisibly for the patient. The apparatus 1 could include a battery 5d which is arranged inside the housing 6, for example. Such an apparatus 1 is especially mobile and can be used everywhere. The measurement takes place in real time and can advantageously be carried out over a longer time period. All data are available in digital form after the measurement. This produces the advantage that a physician can track the extent of the pain during the entire treatment time without any additional effort, with the treatment time being able to last one day or a week or a month, for example. The apparatus in accordance with the invention has the advantage that the instruction to carry out the pain measurement is very simply for the patient because neither the physician nor the patient require a large vocabulary to explain the operation of the apparatus 1 in accordance with the invention and the pain measurement carried out therewith. Previously known pain detection processes require that the patient expresses his pain in words or figures or in units on a scale. The known pain detection processes thus require a cognitive process to translate the felt pain into a unit of measurement. The apparatus in accordance with the invention has the advantage that this cognitive process is not necessary and can thus be bypassed in that the patient intuitively compresses the hollow body of the apparatus 1 in accordance with the invention in accordance with his pain. The apparatus 1 in accordance with the invention is advantageously calibrated before each measurement so that the physician can interpret the pressure measurement or force measurement. The maximum pressure of the hands is measured for the calibration. In an advantageous calibration method, the patient is moreover subjected to differently severe pain stimuli, for example on the back of the free hand which is not holding the apparatus in accordance with the invention, with the aid of a pain actuator 10, for example by generating warmth or heat, and the patient evaluates said pain stimuli by compressing the apparatus 1 in accordance with the invention. The pain actuator 10 is preferably designed as a heat generating device, in particular as a controllable heat generating device, comprising an electric resistor as well as a contact surface which is heated by the electric resistor. In an advantageous calibration method, these calibration data are moreover collected in anonymized form and fed into a central database divided by gender, age, disease and pain medication. From a specific size of the database onward, the physician can compare the measurements carried out using the apparatus 1 in accordance with the invention both with the individual calibration and with the values of the database. It is thereby made possible for a patient to compare his pain with that of other pain patients. The patient can also attempt not to exceed specific pressure values or force values or actually just to reach them in the sense of a therapeutic intervention (biofeedback).

The physician can ask the patient to describe his daily routine and in so doing to state his pain development over this day with the aid of the apparatus 1 in accordance with the invention. The physician can, for example, adjust the pain medication with reference to this recorded pain development in that he e.g. uses a long-term analgesic or prescribes a fast-acting tablet at specific times.

The apparatus in accordance with the invention can be operated in the most varied manner. The data of the pressure sensor or force sensor 2 can, for example, be displayed and stored at a sampling frequency of preferably at least 10 Hz as a curve on the screen of a palm computer of a laptop. A storage via a logger or data memory arranged internally in the apparatus 1 is also possible. In addition to the pressure or to the force, the exact time and/or a marking signal can also be stored. The marking signal, for example, indicates a time in the measurement which the physician wants to look at in more detail later. For example, he triggers the marking in that he clicks the corresponding box of the mask. After the measurement, the values can be shown and printed as a curve or as other functions. To detect and measure pain the patient cannot express verbally, e.g. at the dentist's, a threshold value can be set at the apparatus 1 in accordance with the invention on whose exceeding the apparatus 1 in accordance with the invention optically or acoustically signals to the physician, for example, that the patient is just feeling severe pain.

Although physiological parameters can be strongly influenced by emotional states such as nervousness and fear as well as by medication, it was able to be shown in a study of the applicant that statements can be made on attention and excitement. The simultaneous measurement of the hand warmth, of the perspiration secretion of the hand/or of the heart rate could facilitate or complete the interpretation of the data detected using the apparatus 1 in accordance with the invention. The apparatus 1 in accordance with the invention therefore has additional sensors for detecting the above-named parameters in an advantageous embodiment. It has proved to be particularly advantageous to collect these data, in particular with the additional values of gender, age, disease and/or pain medication, in a large data base, which makes it possible for a physician to compare the behavior of his patient with an average.

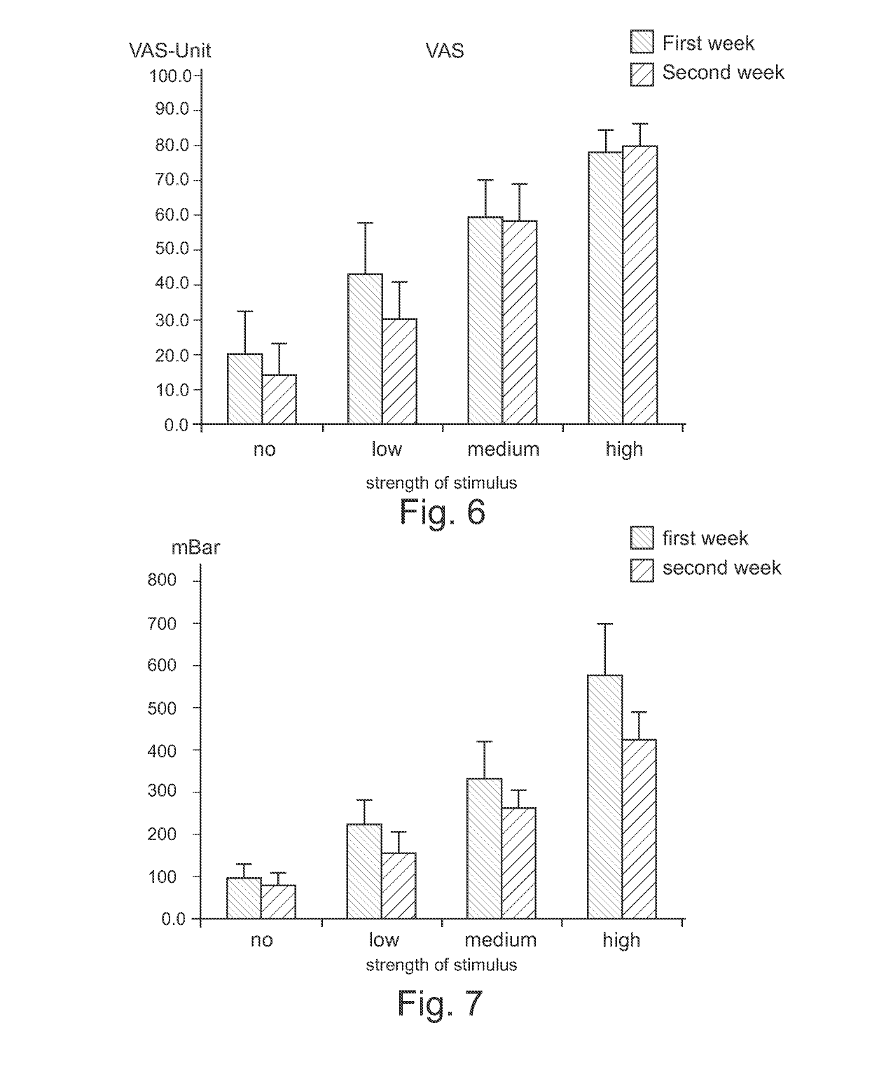

Studies were carried out using the apparatus 1 in accordance with the invention. On the one hand, four different amounts of heat pain stimuli were applied to healthy subjects to the lower arm. They had to assess these using the apparatus 1 in accordance with the invention in the one session and using a visual analog scale, also called VAS, in the other session. To check the reproducibility, the experiment was carried out again exactly one week later. The goal of this study was to check whether the subjects really press more on the respectively stronger stimuli and how the reproducibility is in comparison with VAS. FIG. 7 shows that the apparatus 1 in accordance with the invention is clearly in a position to differentiate the differently strong stimuli. The results of the VAS measurement shown in FIG. 6 likewise differentiate the stimuli, but cannot distinguish the weak stimuli as easily since the measured VAS units of the weak stimuli have a wide spread. This is even though the subjects can see on the basis of the scale visible to them how much they are indicating; in contrast to the apparatus in accordance with the invention with which the subjects do not have any visual feedback. With respect to the reproducibility after one week, the VAS measurement shown in FIG. 6 appears to be better than the measurement in accordance with the invention shown in FIG. 7. Since the circumstances of the different stimuli applied to the subject, however, do not change in the apparatus 1 in accordance with the invention, it is probable that the values of the apparatus 1 in accordance with the invention shown in FIG. 7 rather correspond to the actually felt pain and the reduction in the assessment shown in FIG. 7 represents the accustoming to the stimulus. The results of the apparatus 1 in accordance with the invention shown in FIG. 7 or of the method in accordance with the invention also have a better linearity between the strength of stimulus and the measured pressure or the measured pain in comparison with the VAS measurement shown in FIG. 6. It can also be seen from this that the apparatus in accordance with the invention or the method in accordance with the invention allows pain to be measured more accurately and more reproducibly.

The study shows that patients are able to assess their perceived pain or the pain stimuli exerted on them with high reproducibility using the apparatus in accordance with the invention. An advantage of the apparatus 1 in accordance with the invention can be seen in the fact that it makes it possible to detect very objectively the effect of pain relievers of different strengths on the pain of the patient. The reproducibility of the VAS and NRS is questionable since their results tend to converge or to align when different pain has to be evaluated several times. This convergence does not occur with the apparatus 1 in accordance with the invention. The apparatus 1 in accordance with the invention has the further advantage that the pain can be measured in real time during the occurrence of the pain. The patient can indicate and detect his pain simply and intuitively with the help of the apparatus 1 in accordance with the invention. The patient does not need any feedback for this. This is a very substantial advantage of the apparatus in accordance with the invention in comparison with the VAS method and other methods which are require a visual or verbal feedback or feedback of another kind. Since a number shown visually (VAS) or verbally (NRS, numerical rating scale) can be memorized more easily, whereby it can be remembered easily in later presentations, the intuitive evaluation of the pain using the apparatus 1 in accordance with the invention, which manages without any presentation the user can remember, is very objective. The VAS method has the disadvantage that the memory is included to a great extent in the evaluation of the pain given, which results in the incorrect assessments discovered in the study. It has been shown that older patients, in particular those over 70, do not have any problems in using the apparatus 1 in accordance with the invention.

The known methods in accordance with VAS and NRS have the disadvantage that they require a very good communication between the patient and the physician/carer. If this is lacking, it results in incompletely completed patient files. The apparatus 1 in accordance with the invention and the corresponding method have the further advantage that no measured values are lost. They can thus also be evaluated at a later time. A further advantage can be seen in that the measured values are present in digital form and can therefore also be transmitted to another site with the aid of communication media such as the internet. It is thereby possible also to monitor the pain of a patient remotely in a simple manner. This is, for example, of advantage when the apparatus in accordance with the invention or the method in accordance with the invention is used for the dosing of pain medication. A physician can thus, for example, evaluate the pain situation remotely and prescribe a corresponding dose of the pain medication.

In the most preferred method in accordance with the invention for detecting and measuring the pain of a person, pressure is exerted onto an elastic outer sleeve 3a of a hollow body 3 with the aid of a hand, and in particular by pressing the hand closed, and in so doing a pressure or a force in the hollow body 3 is measured, with the measured pressure or the measured force being used as a measure for the pain perceived by the person.

In a preferred method step, the maximum pressure of the hand is measured before each measurement to detect this maximum force Kₘₐₓ.

In a further preferred method step, a calibration is carried out prior to the measurement in that a patient is subjected to a specific pain, preferably a heat pain, and in that then the pain perceived by the patient is detected by the apparatus in accordance with the invention. A plurality of pain phenomena of different intensities are advantageously applied to the patient, for example sequentially with increasing intensity, to calibrate the measurement. The specific pain applied during the calibration and the measured values of the pain phenomena are advantageously stored in a database.

It can moreover prove to be advantageous to compare the measured pain with the pain values stored in the data base. The measured pain can be compared, for example, with older measurements of the same patient or also with the measured pain of other patients.

In an advantageous method, the pain is measured during a longer time interval, in particular an hour or eight hours or a day, in particular at regular intervals.

In an advantageous method, a pain threshold value is predefined and, if the measured pain exceeds the pain threshold, a signal is generated.

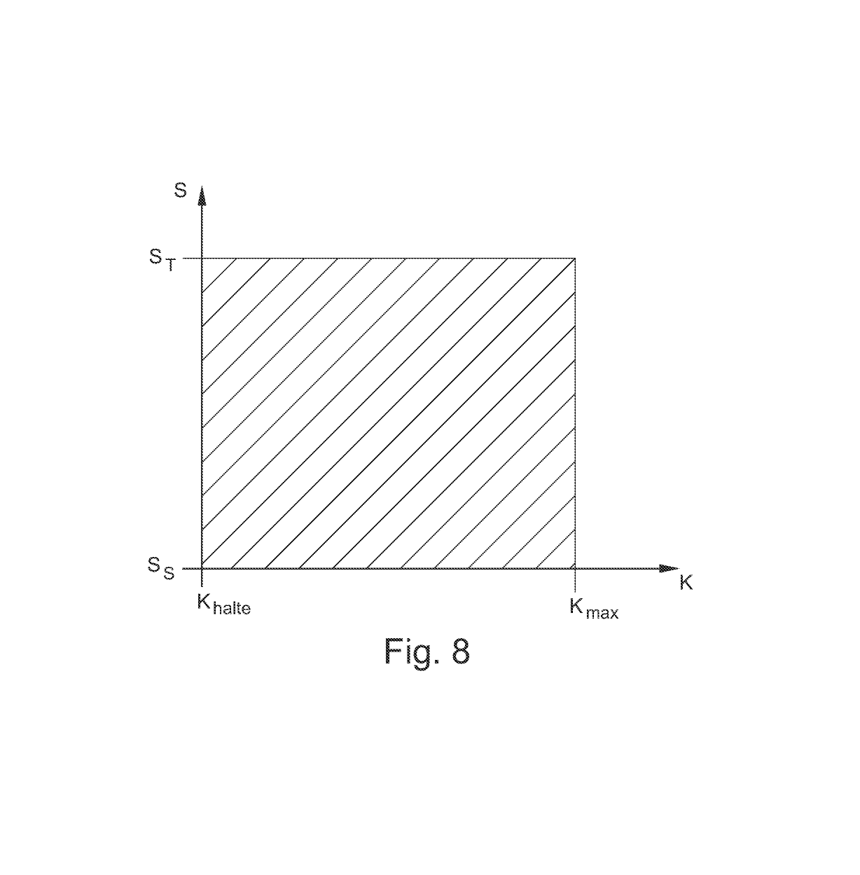

In an advantageous method, the pain measurement is calibrated, as shown in FIG. 8, such that the hand force Kₕₐₗₜₑ required to just hold the apparatus 1 in accordance with the invention tight or to carry it is set as the starting value or zero value. The pain values are advantageously scaled such that the pain threshold value S_{S} at which pain is still just noticeable or is just not yet noticeable, is associated with the hand force Kₕₐₗₜₑ. In addition, the maximum hand force Kₘₐₓ is measured which a patient can generate. The maximum hand force Kₘₐₓ is associated with the maximum tolerable pain value, the so-called pain tolerance value S_{T}. The apparatus 1 in accordance with the invention is thus advantageously verified or calibrated such that the measurement is carried out in the region shown hatched in FIG. 8. The apparatus 1 in accordance with the invention or the method in accordance with the invention can, for example, be used for the dosing of pain medication in that the felt pain is measured in a first step and in that the pain medication is dosed in a subsequent step in dependence on the measured pain and is administered to the patient.

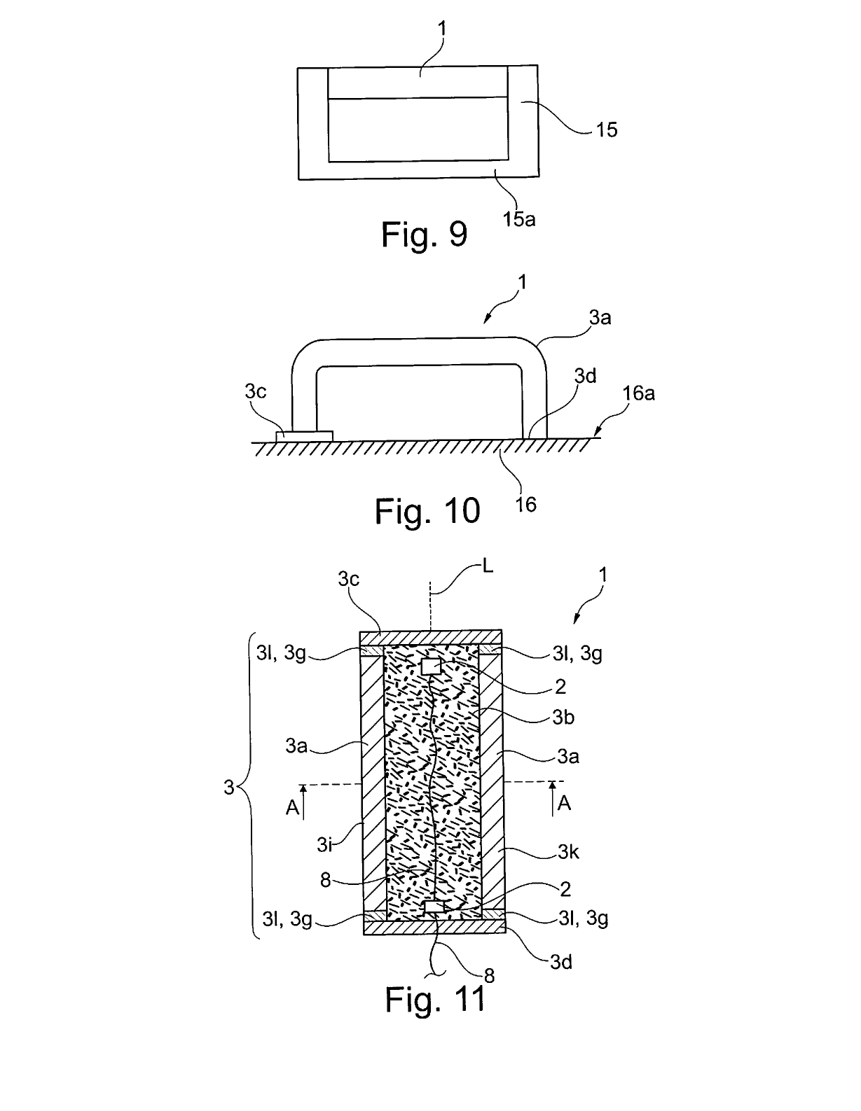

FIG. 9 schematically shows a further embodiment of an apparatus 1 for detecting and measuring pain of a person. The apparatus 1 is connected at its two ends to a holding apparatus 15, with the holding apparatus preferably being designed, as shown, in U shape, with the intermediate space between the apparatus 1 and the base part 15a being designed such that at least the fingers of a hand can be passed through so that the apparatus 1 can be surrounded by a hand, as also shown in FIG. 2. In an advantageous embodiment, the apparatus 1 has, as shown, a cylindrical or a bar-shaped outer contour. The base part 15a can also be designed as a foot which serves to place the apparatus 1 on a base.

FIG. 10 schematically shows a further embodiment of an apparatus 1 for detecting and measuring pain of a person. The apparatus 1 has an outer sleeve 3a extending in U shape and has a respective end part 3c, 3d at either end. The end parts 3c, 3d can, as illustrated on the left with the end part 3c, be formed as a foot so that the apparatus 1 can be placed on the surface 16a of a support 16. The apparatus 1 is designed such that at least the fingers of a hand can be passed through in the intermediate space formed between the apparatus 1 and the support 16 so that the apparatus 1 can be surrounded, as also shown in FIG. 2, by one hand. The end parts 3c, 3d could also, as shown at the right with the end part 3c, be fixedly connected to a further object, for example to the surface 16a of a wall 16. In an advantageous embodiment, the apparatus 1 is designed as battery-operated and has a cableless signal transmission to an external transmission and reception apparatus 12. In an advantageous embodiment, the apparatus 1 has, as shown in FIG. 10, part sections which extend in hollow cylindrical form and which are connected to one another via curved part sections so that the apparatus 1 is designed as a tubular part.

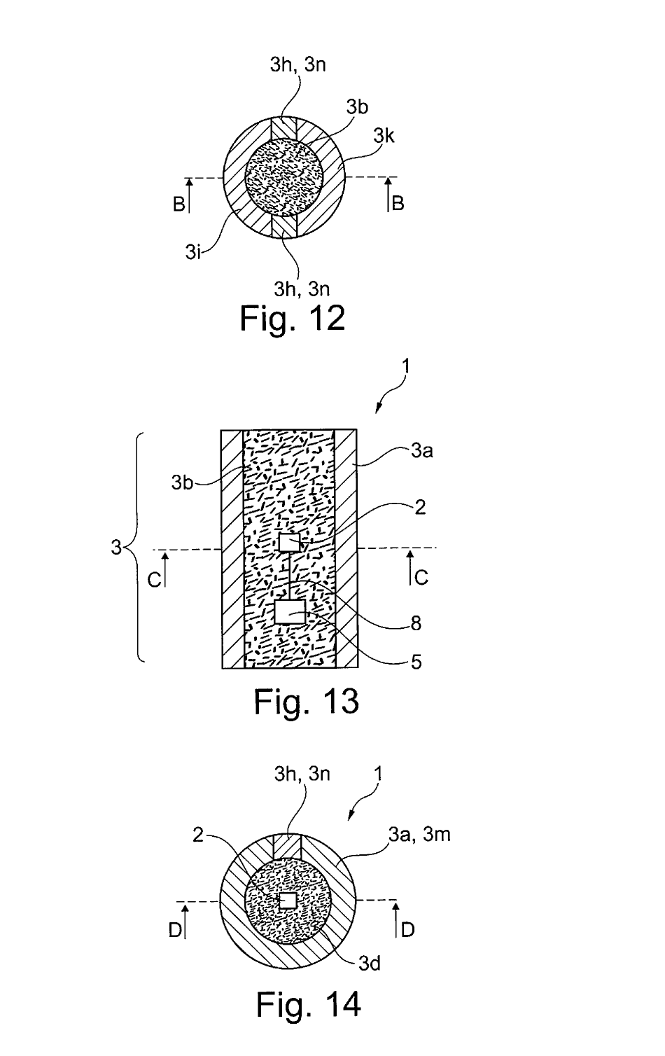

FIGS. 11 and 12 show a further embodiment of a cylindrical or bar-shaped apparatus 1 extending in a longitudinal direction L for detecting and measuring pain of a person, with FIG. 11 showing a longitudinal section along the line B-B and FIG. 12 showing a cross-section along the line A-A. The outer sleeve 3a includes two half-shells 3i, 3k, a first half-shell 3i and a second half-shell 3k, which bound a part of the inner space 3b. The apparatus 1 moreover includes an upper and a lower end part 3c, 3d which in the arrangement shown bound the inner space 3b at the top and at the bottom. The two half-shells 3i, 3k are designed and are arranged in the apparatus 1 such that a force can be introduced into the inner space 3b via the two half-shells 3i, 3k, with the two half-shells 3i, 3k preferably being made as more rigid or having a smaller elasticity than the medium in the inner space 3b. In an advantageous embodiment, the two half-shells have an elasticity smaller by at least a factor of 5 than the material 4a or the fluid 4b in the inner space. In a further possible embodiment, the two half-shells have a greater elasticity, in particular an elasticity greater by at least a factor of 5, or by at least a factor of 10, than the material 4a or the fluid 4b in the inner space. In a preferred embodiment, the two half-shells 3i, 3k are arranged mutually displaceably, preferably only very slightly displaceably. As shown in FIG. 12, the half-shells 3i, 3k have a mutual gap 3h which extends along the whole length of the half-shells 3i. As shown in FIG. 11, the apparatus 1 likewise has a gap 3g between the half-shells 3i, 3k and the upper end part 3c or the lower end part 3d. In a preferred embodiment, an elastic strip 3n of non-flowable material such as rubber or vulcanized rubber is arranged in the gap 3h extending in the direction of the longitudinal axis L. In a preferred embodiment, an elastic ring 31 is likewise arranged in the ring-shaped gap 3g and preferably comprises an elastic, non-flowable material such as rubber or vulcanized rubber. In this embodiment, the inner space is sealed on all sides so that the inner space 3b could also be filled with a liquid fluid. Provided that the inner space 3b is filled with an elastic, non-flowable medium, the longitudinal gap 3h and/or the ring-shaped gap 3g could also be provided with the same medium as the inner space. A pressure sensor or force sensor 2 is arranged in the inner space 3b to measure the force effected on the material in the inner space 3b via the half-shells 3i. In the embodiment shown, a respective sensor 2 is respectively arranged in the end section of the inner space 3b, with the sensors being mutually connected via an electrically conductive cable 8 and being connected to external. The material located in the inner space 3b has a higher elasticity than the two half-shells 3i. The two half-shells 3i can also be designed as very rigid and can, for example, comprise a metal or a hard plastic.

FIG. 13 shows a further embodiment of an apparatus 1 for detecting and measuring pain in a longitudinal section along the line D-D. In comparison with the apparatus 1 shown in FIG. 11, the embodiment shown in FIG. 13 does not have any end part 3c, 3d at the top and bottom, with the inner space 3b being filled with an elastic, non-flowable material such as silicone, rubber or vulcanized rubber or a gel. The cross-section of this embodiment could be designed as shown in FIG. 12 or 14. FIG. 14 shows an embodiment in a cross-section along the line C-C in which the outer sleeve 3a includes a substantially C-shaped spring-elastic part 3m which forms a longitudinal gap 3h between its end sections which is filled with a strip-shaped elastic strip 3n so that the inner space 3b is surrounded in the peripheral direction by the C-shaped shell 3m and by the strip 3n. In an advantageous embodiment, the material located in the inner space 3b has a higher elasticity than the C-shaped shell. The C-shaped shell 3m could also be designed as very rigid and comprise, for example, a metal or a hard plastic. The strip 31 is preferably made more elastic than the spring-elastic shell 3m so that the end sections of the spring-elastic shell 3m are mutually displaceable with a correspondingly engaging force so that a signal can be measured via the pressure sensor or force sensor 2 arranged in the inner space 3b.

The strip 3h could also be provided with the same elastic, non-flowable medium as the inner space 3b. An electronic unit 5 is moreover arranged in the inner space 3b which is connected to the sensor 2 via a cable 8. The electronic unit 5 could, for example, be connected cablelessly to an external apparatus 12. The hollow-cylindrical hollow body 3 preferably has an outer diameter in the range between 1 cm and 5 cm.

All the apparatus 1 in accordance with the invention shown can include a display apparatus 7. In a further advantageous embodiment, the apparatus 1 shown, for example, in FIG. 1 could be designed such that the outer sleeve 3a and the inner space 3b of the hollow body 3 comprise the same material, an elastic, non-flowable material, such as silicone, rubber, vulcanized rubber or a gel. In a possible embodiment, the outer sleeve 3a and the inner space 3b comprise the same material and are preferably designed such that the hollow body 3 comprises a single part with a pressure sensor or force sensor 2 arranged therein or adjacent it.

1. An apparatus (1) for detecting and measuring pain of a person, comprising a pressure sensor or force sensor (2) comprising: a hollow body (3) having an outer sleeve (3a) and an inner space (3b), further comprising an electronic unit (5) that is configured to detect a signal of the pressure sensor or force sensor (2), wherein the outer sleeve (3a) of the hollow body (3) is configured such that it can be surrounded at least partly by a hand, and wherein the inner space (3b) of the hollow body (3) is filled with an elastic, non-gaseous material (4a) or a non-gaseous fluid (4b), and wherein the pressure sensor or force sensor (2) is arranged such as to allow measurement of a pressure of the elastic material (4a) or of the fluid (4b).

2. The apparatus (1) of claim 1, characterized in that the outer sleeve (3a) is elastic, or characterized in that the outer sleeve (3a) includes two half-shells (3i, 3k) which are elastically coupled to one another.

3. (canceled)

4. The apparatus (1) of claim 3, characterized in that the two half-shells (3i, 3k) have an elasticity greater by at least a factor of 5 than the non-gaseous material (4a) or the non-gaseous fluid (4b) in the inner space, and in that the half-shells (3i, 3k) comprise a metal.

5. The apparatus (1) of claim 1, characterized in that the hollow body (3) has a tubular configuration and has a longitudinal axis (L), or characterized in that the pressure sensor or force sensor (2) is located at one end of the hollow body (3).

6. (canceled)

7. The apparatus of claim 2, characterized in that the tubular hollow body (3) is bounded in the longitudinal direction (L) by an upper end part (3c) and a lower end part (3d); and in that the upper end part (3c) has a tubular extension (3e) at whose one end the pressure sensor or force sensor (2) is arranged and whose other end opens into the inner space (3b) of the hollow body (3).

8. The apparatus (1) of claim 7, characterized in that the tubular extension (3e) extends concentrically to the longitudinal axis (L).

9. The apparatus of claim 1, characterized in that the pressure sensor or force sensor (2) is arranged in the interior of the hollow body (3), or characterized in that the hollow body (3) has an elastic outer sleeve (3a) at least at those points which operate as a support for a hand (13), or characterized in that it includes an actuator (10) for generating pain.

10. (canceled)

11. (canceled)

12. The apparatus of claim 10, characterized in that the actuator (10) is controllable by an electronic unit (5).

13. The apparatus of claim 12, characterized in that the electronic unit (5) includes a memory for recording data; and in that the electronic unit (5) includes a read/write apparatus (5c) for a memory card (11).

14. The apparatus of claim 12, characterized in that the electronic unit (5) includes an evaluation unit (5f) which is configured to calculate a pain state of a patient using the measured values of the pressure sensor or force sensor (2).

15. The apparatus of claim 14, characterized in that it includes an optical display (7) which is configured such that the pain state is displayed.

16. The apparatus of claim 14 in accordance with one of the claim 14 or 15, characterized in that it includes an acoustic output apparatus (5g); and in that the acoustic signal, in particular the volume and/or the pitch, are variable in dependence on the pain state.

17. The apparatus of claim 12 in accordance with any one of the preceding claims, characterized in that the electronic unit (5) includes a data transmission apparatus (5c); and in that the data transmission apparatus (5c) allows a data exchange via a cable (8), via radio or via a writing apparatus (5e) for a memory card (11).

18. The apparatus of claim 1, characterized in that it includes an additional sensor (14) for detecting physiological values.

19. (canceled)

20. A method of detecting and measuring pain of a person comprising: providing to a user a hollow body (3) that is manually held by the user; causing the user to exert pressure onto an outer sleeve (3a) of the hollow body (3) by pressing the hand closed, wherein a pressure or a force in the hollow body (3) is measured, and using the measured pressure or the measured force as a measure for the pain perceived by the person.

21. The method of claim 20, characterized in that the outer sleeve (3a) is configured to be shape-stable or substantially shape-stable so that its shape is maintained on the pressing closed of the hand, or characterized in that a maximum pressure of the hand is measured before every measurement, or characterized in that the measurement is calibrated in that a specific pain caused thermally or by heat is applied to the person and in that the pain thereby perceived by the person is measured.

22. (canceled)

23. (canceled)

24. The method of claim 23, characterized in that the determined pain applied on the calibration and the measured pain are stored in a database, and optionally characterized in that the measured pain is compared with pain stored in the database.

25. (canceled)

26. The method of claim 20, characterized in that the pain is measured during a longer time interval, in particular an hour or eight hours or a day, in particular at regular time intervals, or characterized in that a pain threshold value is predefined; and in that, if the measured pain exceeds the pain threshold value, a signal is generated, or characterized in that a hand force (Kₕₐₗₜₑ) which is required to hold the pain measuring device tight or to carry it is set as a starting value or as a zero value.

27. (canceled)

28. (canceled)

29. The method of claim 28, characterized in that a pain threshold value (S_{S}) and a pain tolerance (S_{T}) are determined by an increasingly applied pain.

30. The method of claim 29, characterized in that the pain threshold value (S_{S}) is defined at the value of the hand force (Kₕₐₗₜₑ) which is necessary to hold the pain measuring device tight, or characterized in that the pain threshold value (S_{S}) and the pain tolerance (S_{T}) correlates with the maximum hand force (Kₘₐₓ) in that the pain tolerance (S_{T}) is defined at the level of the maximum hand force (Kₘₐₓ).

31. (canceled)

32. (canceled)
